(19) United States
(12) Patent Application Publication
(10) Pub. No.: US 2013/0296443 A1
(43) Pub. Date: Nov. 7, 2013

(54) SYSTEM AND METHOD OF TRANS-VENOUS PRE-AORTIC GANGLION ABLATION

(71) Applicant: ENIGMA MEDICAL, INC., San Diego, CA (US)

(72) Inventors: Denise Barbut, New York, NY (US); Allan Rozenberg, San Diego, CA (US); Axel Heinemann, New York, NY (US)

(73) Assignee: ENIGMA MEDICAL, INC., San Diego, CA (US)

(21) Appl. No.: 13/787,358

(22) Filed: Mar. 6, 2013

Related U.S. Application Data
(60) Provisional application No. 61/641,599, filed on May 2, 2012.
(60) Provisional application No. 61/724,086, filed on Nov. 8, 2012.
(60) Provisional application No. 61/733,034, filed on Dec. 4, 2012.
(60) Provisional application No. 61/739,396, filed on Dec. 19, 2012.

(51) Int. Cl. A61B 18/14; A61K 31/045; A61K 45/06; A61K 31/05

(52) U.S. Cl. CPC A61B 18/14; A61K 31/05; A61K 31/045; A61K 45/06
USPC 514/731; 514/724; 606/41; 606/33; 606/14; 606/20

(57) ABSTRACT

A method of modulating a physiological parameter of a patient is provided. The method includes disabling one or more pre-aortic ganglion cells within a pre-aortic ganglion trans-venously and improving the physiological parameter. The method further includes destroying a pre-aortic ganglion cell trans-venously to prevent regeneration.

This application claims priority to U.S. Ser. No. 61/641,599, filed on May 2, 2012, and U.S. Ser. No. 61/724,086, filed on Nov. 8, 2012, and U.S. Ser. No. 61/733,034, filed on Dec. 4, 2012, and U.S. Ser. No. 61/739,396, filed on Dec. 19, 2012, the entireties of which are incorporated herein by reference.

### FIELD OF THE INVENTION

The present invention relates generally to the field of hypertension. More specifically, the present invention relates to a system and method of pre-aortic ganglion ablation for the treatment of hypertension.

### BACKGROUND OF THE INVENTION

Hypertension affects tens of millions of individuals. Untreated hypertension is associated with stroke, heart failure and renal failure. Most patients with hypertension are currently treated pharmacologically, many with multiple medications. A quarter of these patients are resistant to medication and their blood pressure poorly controlled, putting them at added risk for complications.

Activation of the sympathetic nervous system is thought to play a significant role in exacerbating hypertension in the later stages of the disease. Reducing such sympathetic activation has been shown to reduce blood pressure in these circumstances.

Recently, mechanical ablation of the renal nerves surrounding the renal artery has been shown to reduce blood pressure in patients with resistant hypertension. The technique consists of an endovascular, arterial procedure and involves radiofrequency ablation of renal nerve fibres, accessed through the wall of the renal arteries bilaterally. Renal artery denervation, as the procedure is known, has been shown to reduce systolic and diastolic pressures by up to 30 mm and 10 mm respectively, and to be persistent out to a year or more following the procedure. The incidence and severity of complications are as yet unknown, as is the long term benefit on blood pressure reduction. Renal nerve fibres regenerate, and the hypotensive effect of this ablative procedure may diminish over time.

Therefore, alternatives to these therapies are needed which provide more significant reductions in blood pressure, persist indefinitely and which are safer, simpler, and less time-consuming.

### BRIEF SUMMARY OF THE INVENTION

The system and method of trans-venous pre-aortic ganglion cell ablation offers a new effective method of controlling blood pressure in patients with medication resistant hypertension. The method in accordance with the invention also overcomes the shortcomings of renal artery denervation. These ganglionic cells can be accessed endovascularly through the vena cava and left renal vein. These methods of treating hypertension have not been previously described.

In one aspect of the invention a method of modulating a physiological parameter of a patient, comprising disabling one or more pre-aortic ganglion cells within a pre-aortic ganglion trans-venously and improving said physiological parameter is provided.

In another aspect of the invention a method of modulating a physiological parameter of a patient is provided, the method comprising trans-venously denervating one or more cells within a pre-aortic ganglion and improving said physiological parameter wherein vessel spasm and dissection are avoided.

In a further aspect of the invention, a method of modulating a physiological parameter of a patient is provided, the method comprising trans-venously denervating one or more cells within a pre-aortic ganglion and improving said physiological parameter wherein deterioration of renal function is avoided.

In a further aspect of the invention, a method of modulating a physiological parameter of a patient is provided, the method comprising trans-venously denervating one or more cells within a pre-aortic ganglion and improving said physiological parameter wherein embolization from a renal artery is avoided.

In a further aspect of the invention, a method of modulating a physiologic parameter of a patient is provided, the method comprising trans-venously denervating one or more cells within a pre-aortic ganglion and improving said physiologic parameter wherein groin hematoma is avoided.

In a further aspect of the invention, a method of modulating a physiologic parameter of a patient is provided, the method comprising trans-venously denervating one or more cells within a pre-aortic ganglion and improving said physiologic parameter wherein femoral artery pseudoaneurysm is avoided.

In a further aspect of the invention a method of modulating a physiologic parameter of a patient is provided, the method comprising trans-venously denervating one or more cells within a pre-aortic ganglion and improving said physiologic parameter wherein groin compression is not required.

### BRIEF DESCRIPTION OF THE DRAWINGS

For a better understanding of the invention, and to show how the same may be carried into effect, reference will now be made, by way of example, to the accompanying drawings, in which:

FIG. 1 is an anatomical depiction of the relationship of the vena cava to the aorta.

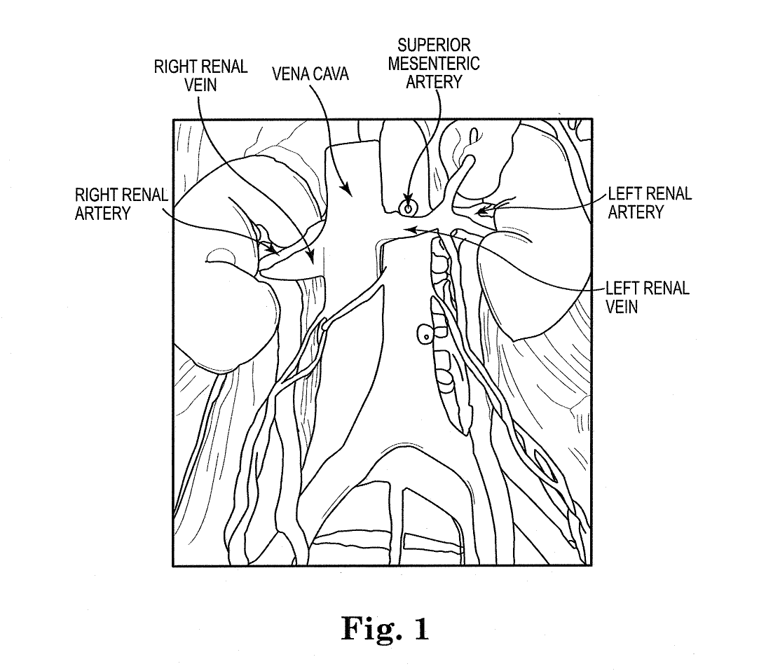

FIG. 2 is an anatomical depiction of the relationship of the vena cava to the aorta.

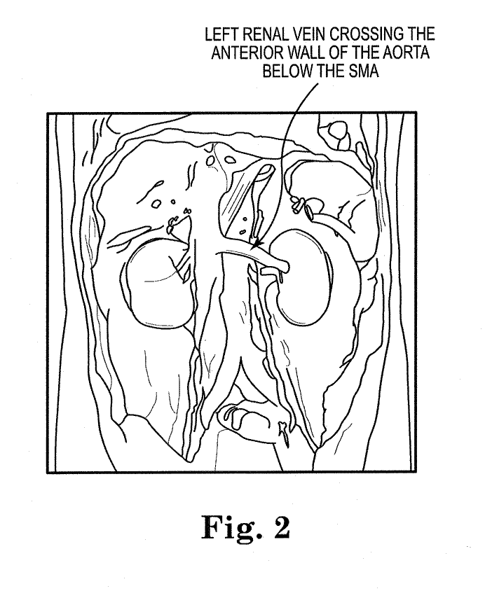

### DETAILED DESCRIPTION OF THE INVENTION

The present invention covers a system and method of trans-venously ablating a portion of the cell bodies within the pre-aortic ganglia for the treatment of hypertension. These can be accessed endovascularly through the vena cava and one of its branches, the left renal vein. The systems and methods of treating hypertension in accordance with the invention have not been previously described.

Hypertension is one of the most common chronic conditions I the world. It affects one in every 7 people globally, or 1 billion people. In the US alone, it affects 1 in 4 adults, close to 70M people. In Europe and Japan, the prevalence is almost double that in the US, affecting 50% or more of adults. It is a major risk factor for heart disease, congestive cardiac failure, stroke and renal failure. The total cost to society was nearly $80 billion in 2010. The risk of death doubles for every 20 mm increase in systolic blood pressure above 120 mm. Conversely, a 5 mm reduction in systolic pressure reduces the risk of stroke by 14%, the risk of heart disease by 9% and the overall mortality by 7%.

Surgical sympathetic denervation for the treatment of resistant hypertension was routinely performed in the 1940's. Such procedures involved removing various combinations of stellate ganglia in the neck, thoraco-lumbar paraspinal ganglia, as well as surgically excising the splanchnic nerve. Blood pressure decreases were very significant, and heart failure was improved. However, such surgical procedures were also associated with significant procedural morbidity and mortality, and were rapidly abandoned in favor of pharmacologic treatments which became available in the 1950's. Pharmacotherapy became the mainstay of management for hypertensive patients during the second half of the last century. Many patients required more than one medication for adequate control of pressure, and up to a quarter of all such patients remained hypertensive on multiple medications (resistant hypertension).

Recently, mechanical means of controlling blood pressure have been revisited, specifically for patients with resistant hypertension. Carotid sinus baroreceptor stimulation using implantable neurostimulation devices has been shown to reduce systolic pressures by up to 40 mm several years after the procedure. The only randomized clinical study using this device missed the primary shorter-term end-point, however, and the study needs to be repeated. Furthermore, procedural complications attributable to the device were high.

Renal artery denervation (RAD) involves ablating renal nerve fibres surrounding renal arteries bilaterally. The procedure involves advancing a catheter endovascularly into each of the renal arteries, and applying ablative energy through the wall of the artery to destroy some of the renal nerve fibres. The treatment lasts about 40 minutes. Procedure related complications are not uncommon. They include, transient bradycardia, embolization from atheromatous renal arteries to kidneys whose function may already be impaired by chronic hypertension, and renal artery spasm or dissection which may also cause deterioration in renal function. While both systolic and diastolic pressures improve following this treatment, the longer term effect on blood pressure is as yet unknown. Peripheral nerve fibres such as those within the renal nerve typically regenerate. Such regeneration following radiofrequency ablation has been demonstrated. After a significant portion of ablated fibres regenerate, the beneficial effect of the procedure on blood pressure may be lost.

In the method in accordance with the invention, the inventors have discovered that denervation of cell bodies rather than nerve fibres may resolve the shortcomings of renal nerve denervation, and furthermore simplify the procedure itself while reducing complications. Unlike nerve axons, cell bodies do not regenerate. Thus following an ablative procedure, destroyed cell bodies do not recover from the insult and are replaced by glial tissue. Any reduction of blood pressure attributable to the procedure is thus likely to be permanent.

The pre-aortic ganglia are located on the antero-lateral aortic wall, many above and below the superior mesenteric artery, closely adherent to the wall of the aorta.

One method of denervating these cell bodies in accordance with the invention includes positioning an ablation device within a vena cava of a patient, advancing it to the level of the superior mesenteric artery and then entering the left renal vein which overlies the ganglia across the anterior aortic wall.

The ablation itself could be performed chemically, using pharmacologic agents, heat or cold, electrical energy or electromagnetic energy such as radiofrequency energy or therapeutic ultrasound, including high frequency focused ultrasound and low frequency ultrasound, or indeed any other technique which would destroy the ganglionic cells. Several parameters may be used to determine further the exact localization of the pre-aortic ganglion cells. By way of example, an energy delivery device may be provided to electrically stimulate the ganglionic cells. Those of skill in the art will appreciate that other similar modes of stimulation may be used and that the energy delivery device may be configured to stimulate or ablate tissue. Lastly, changes in arterial pressure may occur. After the ganglia are localized, the mode may be switched from electrical stimulation to focused ultrasound or to radiofrequency ablation and other modes known to those of skill in the art. Initially, this might cause BP to increase or decrease abruptly. To prevent significant and sudden changes during the procedure and to be able to continuously monitor blood pressure, a pressure sensor may be added to the energy delivery device. The pressure sensor may be configured to feed information back to the energy delivery device and switch it off if blood pressure increased or decreased by more than a predetermined amount.

The most significant advantages of this procedure over pharmacologic treatment alone or renal artery denervation include significantly greater potential reductions in blood pressure and permanence of the hypotension achieved. The extent of the blood reduction achieved is greater because the cell bodies whose axons are destined for the kidney are all very close together. Therefore ablation of even a small area in the relevant portion of the pre-aortic ganglia could destroy large numbers of cells. Indeed, dramatic drops in blood pressure have been reported following pharmacologic pre-aortic denervation in patients with intractable pain secondary to upper abdominal malignancies. In contrast, circumferential ablation of the renal nerve from within the renal artery is likely to destroy only a small fraction of the nerve fibres. Perhaps the most significant benefit of this technique is the permanent nature of the reduction in blood pressure. Destroyed ganglion cell bodies do not regenerate whereas destroyed nerve fibres do regenerate. Dead ganglion cell bodies disappear and are replaced in time by glial tissue. Regeneration of nerve fibres following radiofrequency ablation is well documented. Significant regeneration could lead to the loss of the blood reduction achieved early on following the procedure.

In addition, the inventors have found that this method of treating hypertension is safer, simpler and less time-consuming than renal artery denervation. The vena cava and its branches are thin walled, ensuring adequate contact with and access to the ganglionic cell bodies, The amount of energy required should thus be lower than that required to denervate through a thick arterial wall. Secondly, both right and left ganglion cell bodies can be ablated with a single procedure, as opposed to two procedures, one for each renal artery. Thirdly, problems encountered with renal artery instrumentation do not occur during venous instrumentation. Thus, in 15% of patients who might benefit from renal artery denervation, the renal artery is found to be so stenotic that it cannot be instrumented. Furthermore, instrumented renal arteries frequently go into vasospasm, and may even dissect. Atheromatous material can embolize from the artery to the distal vasculature of the kidney. All of these events can cause further deterioration in renal function in kidneys already compromised by hypertension. Lastly, groin complications following venopuncture are far fewer than those following arterial puncture. Groin hematoma, femoral pseudoaneurysm, time required for groin compression are avoided with procedures involving venipuncture. Although the present invention has been described with reference to preferred embodiments, workers skilled in the art will recognize that changes may be made in form and detail without departing from the spirit and scope of the invention.

We claim:

1. A method of modulating a physiological parameter of a patient, comprising disabling one or more pre-aortic ganglion cells within a pre-aortic ganglion trans-venously and improving said physiological parameter.

2. The method of claim 1 wherein said disabling comprises irreversibly disabling said one or more cells.

3. The method of claim 1 wherein improving said physiologic parameter comprises permanently improving said physiological parameter.

4. A method of modulating a physiological parameter of a patient, comprising destroying a pre-aortic ganglion cell trans-venously to prevent regeneration.

5. The method of claim 4 wherein said physiological parameter is permanently improved.

6. The method of claim 1 or 4 wherein the physiological parameter is associated with heart failure, hypertension, acute myocardial infarction, renal disease, chronic renal failure, obesity, diabetes, ischemic bowel syndrome, obstructive sleep apnea, disorders of intestinal motility, or peripheral vascular disease.

7. The method of claim 1 further comprising trans-venously denervating only a portion of the pre-aortic ganglion including cells that innervate a kidney or an adrenal gland.

8. The method of claim 1 wherein disabling said one or more pre-aortic ganglion cells comprises trans-venously applying an ablative electrical field to said pre-aortic ganglion cells.

9. The method of claim 1 further comprising trans-venously stimulating said pre-aortic ganglion cells; monitoring a physiologic response related to said physiological parameter; trans-venously applying an ablative energy to said one or more pre-aortic ganglion cells; and improving said physiological parameter.

10. The method of claim 1 further comprising providing trans-venous means configured to physically penetrate through the wall of a vein for delivering energy or chemicals directly into said pre-aortic ganglion cells.

11. The method of claim 10 wherein said chemicals are selected from neurolytic agents including phenol, ethanol, anesthetic agents, alpha-blockers, and combinations of the foregoing.

12. The method of claim 9, wherein the physiologic response includes a change in blood pressure.

13. The method of claim 1 wherein pre-aortic ganglion is selected from a celiac ganglion, mesenteric ganglion, suprarenal ganglion, inter-mesenteric ganglion, aortico-renal ganglion, and combinations of the foregoing.

14. The method of claim 1 further comprising providing an energy delivery device; positioning said energy delivery device within a vein directly or proximate a pre-aortic ganglion; and delivering energy through a wall of said vein.

15. The method of claim 14 wherein positioning the energy delivery device within a vein proximate the pre-aortic ganglion comprises positioning the energy delivery device within a vena cava branch to deliver said energy to said pre-aortic ganglion.

16. The method of claim 15 wherein positioning the energy delivery device proximate the pre-aortic ganglion comprises positioning the device within a left renal vein.

17. The method of claim 16 wherein the ablation is performed through the posterior wall of the left renal vein.

18. The method of claim 17 wherein a portion of the left renal vein contacts ganglionic cell bodies of an anterior aortic wall.

19. The method of claim 9 further comprising stimulating the pre-aortic ganglion with an energy delivery device; and monitoring a blood pressure of the patient.

20. The method of claim 17 wherein monitoring said blood pressure includes monitoring a change in said blood pressure.

21. The method of claim 14 wherein delivering energy comprises delivering any wavelength from the electromagnetic spectrum, including radiofrequency, microwave, ultrasound, high intensity focused ultrasound, low intensity focused ultrasound, infrared waves, electrical energy, laser energy, other sources of thermal energy, and combinations of the foregoing.

22. The method of claim 21 wherein said thermal energy comprises cooling.

23. The method of claim 14 wherein a pressure sensor is placed on the energy delivery device.

24. The method of claim 23 further comprising recording the pressure; transmitting said pressure back to the energy delivery device; stopping the ablation if blood pressure increases or decreases within a predetermined parameter.

25. The method of claim 14 wherein said energy delivery device comprises an expandable framework structure or expandable member including one or more electrodes thereon.

26. The method of claim 25 wherein said framework structure or expandable member is cylindrical or spherical.

27. The method of claim 14 wherein said energy delivery device comprises an elongate steerable body including an electrode or transducer thereon.

28. The method of claim 14 wherein said energy delivery device comprises a focused ultrasound device.

29. A method of modulating a physiological parameter of a patient, comprising trans-venously denervating one or more cells within a pre-aortic ganglion and improving said physiological parameter wherein vessel spasm and dissection are avoided.

30. A method of modulating a physiological parameter of a patient, comprising trans-venously denervating one or more cells within a pre-aortic ganglion and improving said physiological parameter wherein deterioration of renal function is avoided.

31. A method of modulating a physiological parameter of a patient, comprising trans-venously denervating one or more cells within a pre-aortic ganglion and improving said physiological parameter wherein embolization from a renal artery is avoided.

32. A method of modulating a physiologic parameter of a patient, comprising trans-venously denervating one or more cells within a pre-aortic ganglion and improving said physiologic parameter wherein groin hematoma is avoided.

33. A method of modulating a physiologic parameter of a patient, comprising trans-venously denervating one or more cells within a pre-aortic ganglion and improving said physiologic parameter wherein femoral artery pseudoaneurysm is avoided.

34. A method of modulating a physiologic parameter of a patient, comprising trans-venously denervating one or more cells within a pre-aortic ganglion and improving said physiologic parameter wherein groin compression is not required.
